(19) United States
(12) Patent Application Publication
(10) Pub. No.: US 2010/0256556 A1
(43) Pub. Date: Oct. 7, 2010

(54) MULTI-COMPARTMENT PACKAGE

(75) Inventors: Stuart J. Cray, JR., Roselle Park, NJ (US); Indradat Jagnandan, Florham Park, NJ (US); David Merle Marxen, Morris Plains, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation Schering Plough Corporation, Kenilworth, NJ (US)

(21) Appl. No.: 12/530,697

(22) PCT Filed: Mar. 27, 2008
(86) PCT No.: PCT/US08/03999

§ 371 Date: Apr. 30, 2010

Related U.S. Application Data
(60) Provisional application No. 60/909,278, filed on Mar. 30, 2007.

(51) Int. Cl. A61M 5/32; B67D 7/78; B65D 81/26; B67D 7/08; A61J 1/14; A61J 7/00; A61M 5/178; A61M 5/00

(52) U.S. Cl. 604/87; 222/145.1; 206/204; 222/153.14; 604/415; 604/93.01; 604/187; 604/272

(57) ABSTRACT

Various aspects of the present invention provide for packages with an outer shell; a first chamber defined at least partially by the outer shell; a second chamber defined at least partially by the outer shell; a fluid-tight rupturable seal separating the first and second chambers; and, a sealed outlet port in communication with the first chamber. With force being applied to the second chamber, the rupturable seal ruptures more readily than the outer shell.

### FIELD OF INVENTION

The present invention is directed to multi-compartment packages. The packages include at least one active pharmaceutical agent.

### BACKGROUND

Drug delivery containers are known in the prior art. Certain active pharmaceutical agents may be highly reactive and unstable or lose efficacy over time when stored in liquid form. These active pharmaceutical agents may be stored in powdered or granular form (e.g., lyophilized) and exposed to a diluent prior to use. The diluent can mixed with the powdered active pharmaceutical agent to reconstitute the active pharmaceutical agent and obtain a active pharmaceutical agent deliverable in flowable form. The powdered and liquid components of such active pharmaceutical agents must be transported and stored in separate containers, however, it would be desirable to house both components in an easy use package.

It may also be desirable to transport certain drugs, such as oxygen sensitive or cytotoxic active pharmaceutical agents in a multicompartment package. Advantageously, such active pharmaceutical agents in a multicompartment package could be mixed with a diluent prior to use.

### SUMMARY OF THE INVENTION

In various aspects of the present invention, there is provided a multi-compartment package with an outer shell; a first chamber defined at least partially by the outer shell; a second chamber defined at least partially by the outer shell; a fluid-tight rupturable seal separating the first and second chambers; and, a sealed outlet port in communication with the first chamber. At least one of said chambers includes at least one active pharmaceutical agent. With force being applied to the second chamber, the rupturable seal ruptures more readily than the outer shell. A moisture sensitive, light sensitive, oxygen sensitive and/or cytotoxic active pharmaceutical agent in powdered or granular form may be used. The package may have a diluent in one chamber. The outer shell may permit visual inspection of said first and/or second chambers.

An overwrap may be provided with the package to form an assembly. The overwrap may include moisture barrier characteristics, gas barrier characteristics, light barrier characteristics, and combinations thereof in providing an addition level of protection for the package.

In a further aspect of the subject invention, a drug delivery system is provided including a package having a first chamber, a second chamber, and a fluid-tight seal separating the first and second chambers which ruptures more readily under application of force than other portions of the package. A first active pharmaceutical agent may be disposed in the first chamber. The first component located in the first chamber may be a liquid or a non-liquid such as a solid such as a dry powder such as a lyophilized powder or granular form. A second component is disposed in the second chamber. The second component may be a liquid or non-liquid. Suitable second components include flowable products such as a liquid and may include an active pharmaceutical agent. The liquid may be solution or a suspension. The seal can be caused to rupture at point of use with the first and second components coming into contact and mixing to form an administrable active pharmaceutical agent.

In a further aspect of the subject invention, a drug delivery system including a package having a first chamber, a second chamber, and a fluid-tight seal separating said first and second chambers. Preferably, the seal ruptures more readily under application of force than other portions of said package. A first component disposed in the first chamber may include a liquid or a non-liquid. The first component may include an active pharmaceutical agent. Either or both components may be a liquid or a non-liquid. Either or both components may be a solution or suspension. The first or second component or both may include one or more active pharmaceutical agents. Either component may be a powder, granule, film, bead, wafer and combinations of two or more thereof. Advantageously, at least one cytotoxic, moisture sensitive or oxygen sensitive active pharmaceutical agent may be contained in the same package with a diluent.

Other embodiments of the present invention provide a multi-compartment package which includes an outer shell; a first chamber defined at least partially by the outer shell; wherein the first chamber includes a non-liquid such as a solid; a second chamber defined at least partially by the outer shell; and a fluid-tight rupturable seal separating the first and second chambers. A sealed outlet port may be in communication with the first chamber.

In various embodiments, the second chamber includes a liquid. Each of the chambers may include at least one active pharmaceutical agent. The at least one active pharmaceutical agent may include a cytotoxic agent or the at least one active pharmaceutical agent could be light sensitive, moisture sensitive, oxygen sensitive or highly reactive or a combination of two or more. The at least one active pharmaceutical agent may be highly reactive with other excipients or other active pharmaceutical agents.

Other embodiments provide for a multi-compartment package including an outer shell; a first chamber defined at least partially by the outer shell; wherein the first chamber comprises a non-liquid, such as a solid; a second chamber defined at least partially by the outer shell; wherein the second chamber comprises a liquid; wherein each of the chambers comprises at least one active pharmaceutical agent; a fluid-tight rupturable seal separating the first and second chambers; and, a sealed outlet port in communication with the first chamber, wherein, with force being applied to the second chamber, the rupturable seal ruptures more readily then the outer shell.

At point of use, the seal separating the contents of the two chambers may be ruptured to permit the diluent to mix with the powdered active pharmaceutical agent and cause reconstitution thereof. The reconstituted active pharmaceutical agent may then be administered through the outlet port.

These and other features of the invention will be better understood through a study of the following detailed description and accompanying drawings.

### BRIEF DESCRIPTION OF THE DRAWINGS

FIG. 1 is a plan view of a package formed in accordance with various embodiments of the present invention;

FIG. 2 depicts a stopper as a seal for the outlet port of the package; and,

FIG. 3 is a schematic of an assembly usable for forming a package in accordance with various embodiments of the present invention.

### DETAILED DESCRIPTION OF THE INVENTION

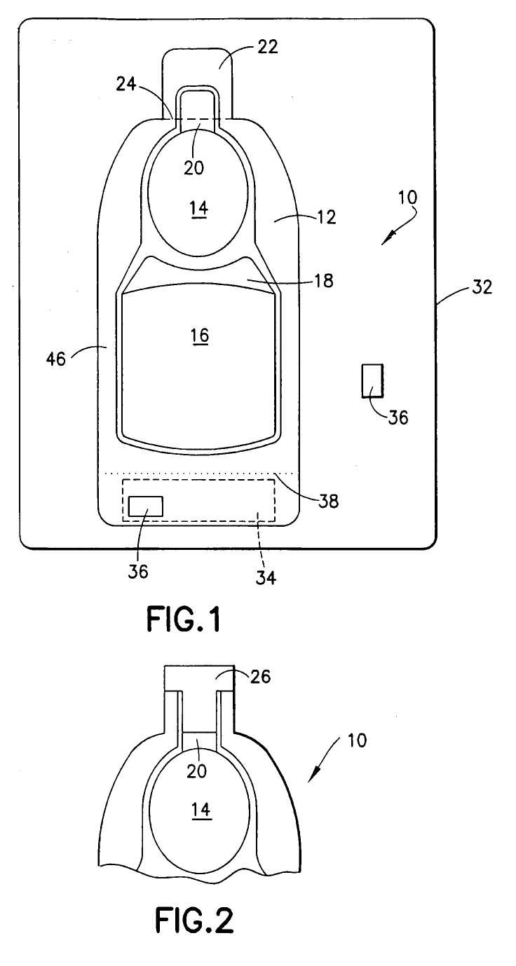

With reference to FIG. 1, a multi-compartment package 10 is depicted. The package 10 includes an outer shell 12 which at least partially defines a first chamber 14 and a second chamber 16. A fluid-tight rupturable seal 18 separates the first and second chambers 14, 16. The package 10 may be provided with any configuration, with the first and second chambers 14, 16 being sized as needed, as described more fully below.

The first chamber 14 and the second chamber 16 may accommodate a two-part medication. In various embodiments, the first chamber 14 accommodates a powdered component, with the second chamber 16 accommodating a diluent or medication in flowable form, such as a liquid (e.g., a syrup) or slurry. In other embodiments, the first and second chamber includes a liquid component. In other embodiments, the first chamber accommodates a liquid component and the second chamber accommodates a powder or non-liquid component. The accommodated components may or may not include active pharmaceutical agents or medicaments. A liquid component may contain a solution or a suspension. The first and second chambers 14, 16 may be sized to accommodate sufficient quantities of the two-part medication to provide a single unit dose. Suitable non-liquids include solids including but not limited to, powders, granules, films, beads, wafers and combinations of two or more thereof.

Many active pharmaceutical agents may be sensitive to light, moisture, gases, or interact with other active pharmaceutical agents, etc. Sensitivity to these factors may cause the agents to be instable and possible to degrade. Various aspects of the present invention advantageously provide a unit or single dosage form of one or more active pharmaceutical agents (APAs). Such dosage forms are advantageously capable of having a consistent delivery with acceptable content uniformity that is required for pharmaceutical products. Another advantage is that various embodiments are capable of providing a way to reconstitute active pharmaceutical agents that need to be stored in a dry, non-liquid form with a liquid form in an enclosed environment. This enclosed environment is less likely to lose any parts, e.g. powder or particles, such as may be the case if a powder is mixed in an open air environment. This is a distinct advantage for pharmaceutical products where content uniformity of a dose is of great importance. The enclosed environment may be sterile, which provides further advantages. Additionally, some APAs may be cytotoxic. Reconstituting an APA in a closed environment lowers the likelihood that some of the APA could contaminate the open air environment and be ingested or inhaled by someone other than the patient in need of such APA. Suitable at least one active pharmaceutical agents include but are not limited to, cytotoxic, light sensitive, moisture sensitive, oxygen sensitive, highly reactive agents and combinations thereof.

By way of non-limiting example, the first chamber 14 may accommodate a dry montelukast sodium granule or powder medication, and the second chamber 16 may accommodate a liquid such as syrup including loratadine, cetirizine, fexofenadine or desloratadine. As a further example, the first chamber 14 may accommodate a toxic active pharmaceutical agent, such as a temozolomide, and the second chamber 16 may accommodate sterile liquid such as water. Other suitable APAs include but are not limited to interferon.

As will be appreciated by those skilled in the art, the package 10 may include additional chambers to accommodate three- or more part medications.

Suitable liquids for the compartments include diluents such as but are not limited to water, sterile water, and the like. Liquids may also contain additives such as sweeteners, preservatives, antioxidants, chelating agents, thickeners, and the like. Combinations of two or more additives may be included.

To permit administration of a prepared dose of medicine, an outlet port 20 is provided, preferably in communication with the first chamber 14. The outlet port 20 may be sealed by a portion 22 of the outer shell 12. A frangible line of weakness 24 may be provided to facilitate detachment of the portion 22 in exposing the outlet port 20 for administration. Alternatively, as shown in FIG. 2, a stopper or other closure member 26 may be provided to seal the outlet port 20. The other closure member may be opened by tearing either by a sharp object such as a scissors or by providing a notch so that it may be manually broken or torn open. The stopper 26 may be formed of an elastomeric or other piercable material which permits a medical injector to pass therethrough and withdraw reconstituted active pharmaceutical agent from the first chamber 14. The outlet port 20 may include an attachment means to enable attachment of an object or device to the outlet port. Suitable attachment means include a luer lock, which can allow the attachment of a desirable delivery device such as a needle, syringe, oral dose dispenser, oral metered dose dispenser or applicator.

The outlet port 20 may include a valve such as a spring valve or the like. The amount of liquid located in the compartments dispensed through an outlet port including a valve can be controlled. Advantageously, valves can be utilized such that a desired dose or amount is delivered into a delivery vehicle that can be attached to the package 10. A specific dosage may be delivered from the delivery vehicle to a patient. Thus, this system or package can be used for different dosing strengths that can accommodate different patients.

Suitable delivery vehicles include a needle, syringe, oral dose dispenser, oral metered dose dispenser or applicator. A valve, such as a spring valve, can facilitate a specific amount or dose to be delivered into a delivery vehicle. The delivery vehicle can include measuring markers so that it can be utilized to measure a desired specific metered or measured dosage amount. The delivery vehicle may be permanently attached to the outlet port or detachable to the outlet port. Detachable delivery vehicles may be attached to the package via a suitable attachment means such as a luer lock.

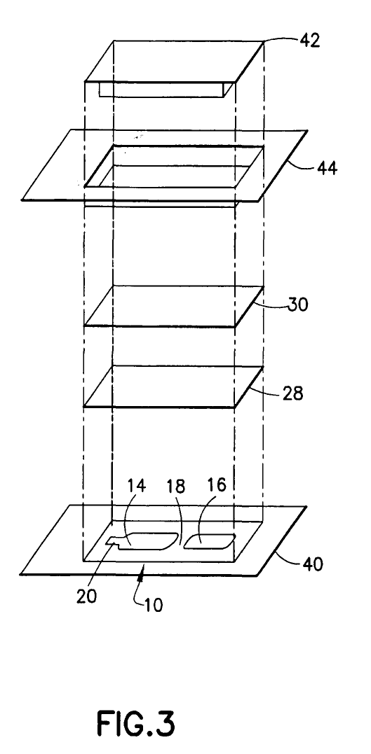

The package 10 may be formed as a rigid or flexible package in the form of a blister package or a pouch/sachet package. As will be appreciated by those skilled in the art, any technique may be used to form the package 10. It is preferred that the package 10, particularly the outer shell 12, be formed of material having gas and/or moisture barrier characteristics. By way of non-limiting example, the outer shell 12 may be formed by first and second sheets 28, 30 which are secured together, as shown in FIG. 3.

Preferably, the first and second sheets 28, 30 are secured at discrete locations. The first and second sheets 28, 30 may be secured together using any technique, including, but not limited to, fusing and/or bonding. The first and second sheets 28, 30 may define the entirety of the package 10, including the outer shell 12, the first and second chambers 14, 16, and the rupturable seal 18.

One or both of the first and second sheets 28, 30 may be formed from a single ply or laminated structure of elastomers, thermoplastics such as polyolefins, aluminum foil and combinations of two or more thereof. The sheets may be formed with at least one barrier including moisture resistant barrier, gas resistant barrier, light resistant barrier (e.g., to preserve light sensitive active pharmaceutical agent products), odor resistant barrier and combinations of two or more thereof. Suitable materials include at least one of cyclic olefin copolymers, ethylvinyl acetate, polyethylene, polyester, polychlorotrifluoroethylene (PCTFE), polyvinyl chloride, aluminum foil, and combinations of two or more thereof. The first sheet 28 and the second sheet 30 may be formed of the same or different materials. It is preferred that at least one of the first and second sheets 28, 30 be formed clear to allow visual inspection of the first and/or second chamber 14, 16. Additionally, all materials forming the package 10 may be sterilizable.

With reference to FIG. 1, as additional protection for the package 10, an overwrap 32 may be provided which can be a pouch or folded sheet formed to encompass the entirety of the package 10. The overwrap 32 may be formed with at least one barrier including moisture resistant barrier, gas resistant barrier, light resistant barrier (e.g., to preserve light sensitive active pharmaceutical agent products), odor resistant barrier and combinations of two or more thereof. The barriers may be formed of the same materials as noted above for the first and second sheets 28, 30. The overwrap may include a material including antioxidants, odor absorbers, moisture absorber, off-gassing absorbers, relative humidity maintaining level materials and combinations of two or more thereof. Suitable materials include, dessicants such as silica gel and atomic sieve particles. The overwrap 32 may be notched to facilitate removal thereof. The overwrap 32 provides an additional level of protection for the package 10, with the collective assembly being possibly considered a child-resistant package.

In addition, the package 10 may be provided with a third chamber 34 for accommodating a packet of third material 36 such as a desiccant or other moisture absorbing material. Suitable third materials antioxidants, odor absorbers, moisture absorber, off-gassing absorbers, relative humidity maintaining level materials and combinations of two or more thereof. Suitable third materials include, dessicants such as silica gel and atomic sieve particles. A desiccant acts to absorb moisture in minimizing moisture exposure to any accommodated dry active pharmaceutical agent. The third chamber 34 is preferably completely encompassed in the package 10 and sealed from the first and second chambers 14, 16. In addition, a line of weakness 38, such as perforations, may be defined in the package 10 to permit access to the third chamber 34 and removal of the third material 36. It may be preferred to remove the desiccant prior to mixing of the active pharmaceutical agent components. Alternatively, the packet of desiccant may be disposed in the overwrap 32 externally of the package 10.

Using any known technique, the rupturable seal 18 is formed so as to rupture more readily than the outer shell 12. For example, the rupturable seal 16 may be defined as a bonded region between the first and second sheets 28, 30. The level of strength of the bond is preferably set below the rupture strength of either of the first or second sheets 28, 30. In this manner, pressure applied to one or both of the first and second chambers 14, 16 will result in rupture of the rupturable seal 18 rather than rupturing of the first and second sheets 28, 30.

The package 10 may be stored and transported under similar circumstances as other active pharmaceutical agent containers, e.g., being refrigerated, shelf life, etc. Once at point of use, the package 10 may be prepared for administration. The overwrap 32 and the desiccant may be initially removed. Thereafter, pressure may be applied to one or both of the first and second chambers 14, 16 so as to cause rupture of the rupturable seal 18. It is preferred that pressure only be applied to the chamber accommodating the flowable product (e.g., liquid) which, preferably, is the second chamber 16. It is further preferred that the seal for the outlet port 20 remain intact even with rupturing of the rupturable seal 18. In this manner, the active pharmaceutical agent components do not leak out prematurely. Once the seal 18 has been ruptured, the two components are able to come into contact and mix. The package 10 may be shaken to enhance the mixing effect. With one or both of the sheets 28, 30 being clear, visual inspection of the mixing of the two components is possible. With sufficient mixing, the dry active pharmaceutical agent is reconstituted and ready for administration. The seal to the outlet port 20 is removed or breached (e.g., by a medical injector) and the mixed liquid may be administered orally or otherwise (e.g., by injection).

With reference to FIG. 3, a representative method of preparing the package 10 is depicted. The method utilizes a support platen 40 arranged to cooperate with first and second sealing platens 42, 44. Additional support and sealing platens may be utilized. The first and second sealing platens 42, 44 are preferably configured to provide different levels of bonding to the first and second sheets 28, 30, such as by applying different levels of heat, dwell time, and/or pressure. It is preferred that periphery 46 of the package 10 be formed with a seal of high integrity with a rupture threshold higher than that of the rupturable seal 18. The first seal platen 42 may be configured to cause bonding between the first and second sheets 28, 30 to define the rupturable seal 18, while the second platen 44 may be configured to cause bonding between the first and second sheet 28, 30 to define the periphery 46. With this arrangement, the second platen 44 would provide more heat, dwell time and/or pressure in defining a stronger bond than the first platen 42. It is preferred that although the rupturable seal 18 may more readily fail than other portions of the package 10, the rupturable seal 18 preferably still provides a fluid tight seal.

The components may be placed into the first and second sheets 28, 30 prior to sealing. Optionally, the first and second sheets 28, 30 may be partially bonded, e.g. with discontinuations being left in the periphery 46, with the components being loaded through the formed openings, including the inlet port 20. The first and second sheets 28, 30 may be then further sealed to complete the package 10. Any known technique can be used to define the chambers 14, 16, 34 and the inlet port 20 between the first and second sheets 28, 30.

The foregoing descriptions of various embodiments of the invention are representative of various aspects of the invention, and are not intended to be exhaustive or limiting to the precise forms disclosed. Many modifications and variations may occur to those having skill in the art. It is intended that the scope of the invention shall be fully defined solely by the appended claims.

What is claimed is:

1. A multi-compartment package comprising:
an outer shell;
a first chamber defined at least partially by said outer shell;
a second chamber defined at least partially by said outer shell; wherein at least one of said chambers comprises at least one active pharmaceutical agent;
a fluid-tight rupturable seal separating said first and second chambers; and,
a sealed outlet port in communication with said first chamber,
wherein, with force being applied to said second chamber, said rupturable seal ruptures more readily then said outer shell.

2. A package as in claim 1, wherein said outlet port is sealed with a stopper.

3. A package as in claim 1, wherein said outlet port is sealed by a portion of said outer shell.

4. A package as in claim 1, wherein said outer shell comprises at least one barrier selected from the group consisting of a moisture resistant barrier, gas resistant barrier, light resistant barrier, odor resistant barrier and combinations of two or more thereof.

5. A package of claim 1, wherein said at least one active pharmaceutical agent is selected from the group consisting of montelukast, loratadine, desloratadine, cetirizine, fexofenadine, temozoiomide, interferon and combinations of two or more thereof.

6. A package as in claim 1, wherein said outer shell includes at least one material selected from the group consisting of elastomers, thermoplastics, aluminum foils and combinations of two or more thereof.

7. A package as in claim 1, wherein said outer shell includes at least one material selected from the group consisting of polyethylene, polyester, polychlorotrifluoroethylene (PCTFE), polyvinyl chloride, aluminum foil, and combinations of two or more thereof.

8. A package as in claim 1, wherein said outer shell permits visual inspection of said first and second chambers.

9. A package as in claim 1, wherein said package includes a third chamber sealed from said first or second chambers.

10. A package as in claim 9, wherein said third chamber comprises at least one material selected from the group consisting of antioxidants, odor absorbers, moisture absorber, off-gassing absorbers, relative humidity maintaining level materials and combinations of two or more thereof.

11. A package as in claim 9, wherein a line of weakness is defined in said outer shell to permit access to said third chamber.

12. A package as in claim 1, wherein said rupturable seal ruptures more readily than the seal of said outlet port.

13. A package of claim 1, wherein the outlet port comprises a luer lock.

14. A package of claim 1, wherein a delivery vehicle is connected to the outlet port.

15. A package of claim 1, wherein the delivery vehicle is connected to the outlet port via a luer lock.

16. A package of claim 14, wherein the delivery vehicle is selected from the group consisting of a needle, syringe, oral dose dispenser, oral metered dose dispenser or applicator.

17. An assembly comprising:
a package of claim 1; and,
an overwrap disposed about said package.

18. An assembly as in claim 17, wherein said overwrap comprises at least one material selected from the group consisting of antioxidants, odor absorbers, moisture absorber, off-gassing absorbers, relative humidity maintaining level materials and combinations of two or more thereof.

19. An assembly of claim 17, wherein said overwrap is formed with at least one barrier selected from the group consisting of a moisture resistant barrier, gas resistant barrier, light resistant barrier, odor resistant barrier and combinations of two or more thereof.

20. A drug delivery system comprising:
a package having a first chamber, a second chamber, and a fluid-tight seal separating said first and second chambers, said seal rupturing more readily under application of force than other portions of said package;
a first component disposed in said first chamber, and
a second component disposed in said second chamber, wherein at least one component comprises at least one pharmaceutical active agent.

21. A system as in claim 20, wherein said second component is in a liquid or slurry form.

22. A system as in claim 20, wherein said second component is a solution or suspension.

23. A system as in claim 20, wherein said second component includes at least one active pharmaceutical agent.

24. A system as in claim 20, wherein said first component includes at least one active pharmaceutical agent.

25. A system as in claim 20, wherein said first component is in a liquid or slurry form.

26. A system as in claim 20, wherein said first component is in a non-liquid form.

27. A system as in claim 20, wherein said first component is selected from the group consisting of a powder, granule, film, bead, wafer and combinations of two or more thereof.

28. A system of claim 20, wherein said package further comprises a sealed outlet port in communication with the first chamber.

29. A system of claim 28, wherein the outlet port comprises a luer lock.

30. A system of claim 28, wherein a delivery vehicle is connected to the outlet port.

31. A system of claim 30, wherein the delivery vehicle is connected to the outlet port via a luer lock.

32. A system of claim 30, wherein the delivery vehicle is selected from the group consisting of a needle, syringe, oral dose dispenser, oral metered dose dispenser or applicator.

33. A multi-compartment package comprising:
an outer shell;
a first chamber defined at least partially by said outer shell; wherein said first chamber comprises it non-liquid;
a second chamber defined at least partially by said outer shell; wherein said second chamber comprises a liquid;
wherein each of said chambers comprises at least one active pharmaceutical agent;
a fluid-tight rupturable seal separating said first and second chambers; and,
a scaled outlet port in communication with said first chamber,
wherein, with force being applied to said second chamber, said rupturable seal ruptures more readily then said outer shell.

34. A multi-compartment package as in claim 33, wherein the at least one active pharmaceutical agent is selected from the group consisting of cytotoxic, light sensitive, moisture sensitive, oxygen sensitive, highly reactive agents and combinations thereof.

35. A multi-compartment package as in claim 1, wherein the at least one active pharmaceutical agent is selected from the group consisting of cytotoxic, light sensitive, moisture sensitive, oxygen sensitive, highly reactive agents and combinations thereof.
